(19) United States
(12) Patent Application Publication
(10) Pub. No.: US 2004/0039202 A1
(43) Pub. Date: Feb. 26, 2004

(54) Cationic dyes, method for the production thereof, and colouring agents containing said compounds

(76) Inventors: Guido Sauter, Thoerishaus (CH); Hans-Juergen Braun, Ueberstorf (CH); Raymond Brouillard, Strasbourg (FR); Andre Fougerousse, Birkenwald (FR); Christine Roehri-Stoeckel, Strasbourg (FR); Emmanuel Gonzalez, Dijon (FR)

Correspondence Address: Striker Striker & Stenby, 103 East Neck Road, Huntington, NY, 11743, US

(21) Appl. No.: 10/381,919

(22) PCT Filed: Feb. 7, 2002
(86) PCT No.: PCT/EP02/01268

(30) Foreign Application Priority Data
Jun. 22, 2001 (DE) 101 30 145.6

(51) Int. Cl. C 07D 4 5/02; C07D 491/02

(52) U.S. Cl. 544/333; 546/282.7; 548/266.4; 549/387; 549/398; 549/218

(57) ABSTRACT

The present invention concerns compounds of formula (V) and (VI), a method for producing them and colorants containing compounds of formula (V) and/or (VI).

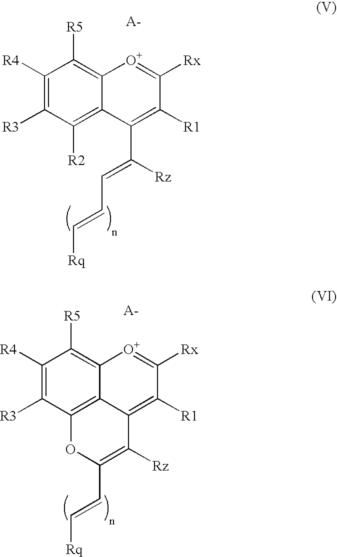

The object of the present invention are novel cationic dyes, a method for producing them and dye compositions containing these cationic dyes.

Different methods of synthesizing certain 2-alkylene-2-aryl-4H-1-benzopyran derivatives and their salts are already known from the literature, see, for example, J. G. Sweeney and G. A. Jacobucci in Tetrahedron 37, 1481-1483 (1981), G. A. Jacobucci and J. G. Sweeney in Tetrahedron 39, 3005-3038 (1983) and M. Elhabiri, P. Figuereido, A. Fougerousse and R. Brouillard in Tetrahedron Lett. 36, 4611-4614 (1995). A frequently used method is, for example, the condensation reaction between a 2-hydroxybenzaldehyde derivative and an acetophenone derivative which affords a 4-alkylene-2-aryl-4H-1-benzopyranderivative. Another method involves the reaction of a reducing agent or an oxidant with diverse flavonoids. These methods, however, are not satisfactory in every respect.

Surprisingly, we have now found that 4-alkylene-2-aryl-4H-1-benzopyran derivatives of formula (Ia) or their salts of formula (I), which can be prepared in simple manner by reaction of a flavone of formula (II) with a Grignard compound of formula (III), are eminently suited as starting materials for the preparation of novel cationic dyes of formulas (V) and (VI).

The 4-alkylene-2-aryl-4H-1-benzopyran derivatives of formula (Ia) or their salts of formula (I) serving as starting materials are prepared by reaction of one mole of a flavone of formula (II) in an apolar aprotic or polar aprotic solvent with 1 to 50 moles of a compound of formula (III) at a temperature of −80° to 180° C. followed by treatment of the reaction product with an acidic, aqueous or aqueous-alcoholic solution;

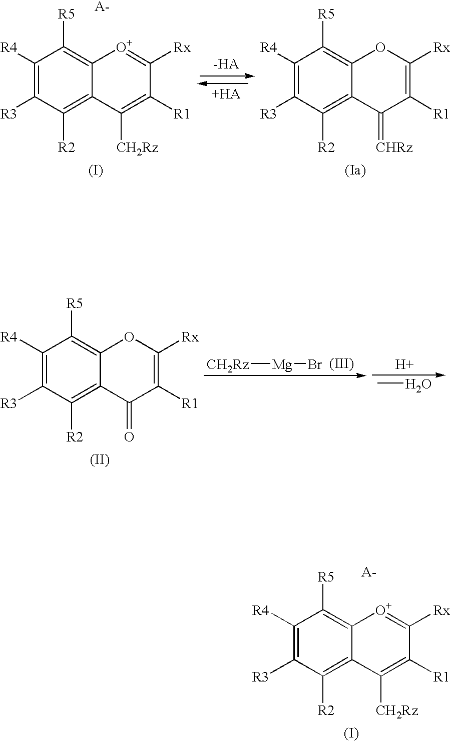

wherein

R1, R2, R3, R4 and R5 independently of each other denote a hydrogen atom, a straight-chain or branched C₁-C₄-alkyl group, a C₁-C₄-hydroxyalkyl group, a hydroxyl group, a methoxy group, a benzyl group, a halogen atom (F, Cl, Br, I), a nitro group, a nitroso group, a cyano group, a trifluoromethyl group, a —CHO group, a —COR^{b} group, a —COOH group, a —CO₂R^{b} group, an —OCOR^{b} group, an —OCH₂-aryl group, an —SO₂NH₂ group, an —SO₂CHF₂ group, an —SO₂CF₃ group, an —SO₂NH₂ group, an —SO₂NHR^{b} group, an —SO₂N(R^{b})₂ group, an —SO₂R^{b} group, an —NH₂ group, an —(NH₃)⁺ group, an —NHR^{b} group, an —(NH₂R^{b})⁺ group, an —N(R^{b})₂ group, an —[N(R^{b})₃]⁺ group, an —NHCOR^{b} group, an —NHCOOR^{b} group, a CH₂NH₂ group, a —CH₂NHR^{b} group, a —CH₂N(R^{b})₂ group, a —CO₂CF₃ group or a —PO(OR^{b})₂ group, wherein R^{b} stands for a hydrogen atom, an optionally substituted aromatic carbon ring or heterocyclic ring or a (C₁-C₆)-alkyl group;

Rx denotes a hydrogen atom, an optionally substituted phenyl group, an optionally substituted naphthyl group, a benzyl group, an optionally substituted aromatic carbon ring or heterocyclic ring, a C₁-C₈-polyhydroxyalkyl group, a C₁-C₈-alkoxy-(C₁-C₈)-alkyl group, a straight-chain or branched C₁-C₈-alkyl group, an —OR^{a} group or an SR^{a} group;

Rz denotes a hydrogen atom, an optionally substituted phenyl group, an optionally substituted naphthyl group, an optionally substituted aromatic carbon ring or heterocyclic ring, a C₁-C₈-alkyl group, a C₁-C₈-monohydroxyalkyl group, a C₁-C₈-polyhydroxyalkyl group, a C₁-C₈-alkoxy-(C₁-C₈)-alkyl group, a halogen atom, an —OCOR^{a} group, a nitro group, a cyano group, a —CO—R^{a} group, a —CO—OR^{a} group, a —CO—OCF₃ group, a —CONHR^{a} group, a —CO—N(R^{a})₂ group, an —SO₂—NH₂ group, an —SO₂—NHR^{a} group, an —SO₂—N(R^{a})₂ group, an —SO₂—OR^{a} group or an —SO₂R^{a} group wherein R^{a} stands for a hydrogen atom, an optionally substituted aromatic carbon ring or heterocyclic ring or a C₁-C₆-alkyl group;

A′ denotes the anion of an organic or inorganic acid, preferably a chloride, bromide iodide, hydrogen sulfate, sulfate, toluenesulfonate, benzenesulfonate, monomethyl sulfate, hexafluorophosphate, hexafluoroantimonate, tetrafluoroborate, tetraphenylborate, formate, acetate or propionate, with the chloride ion, tetrafluoroborate ion, acetate ion and hydrogen sulfate ion being particularly preferred.

The Grignard reaction between compounds of formula (II) and formula (III) is carried out at −80° to 180° C., preferably at 0° to 140° C. and particularly at 200 to 100° C., In an apolar aprotic or polar aprotic solvent, for example an ether, preferably diethyl ether, dioxane, 1,2-dimethoxyethane, 1,2-diethoxyethane, bis-(2-ethoxyethyl) ether and particularly tetrahydrofuran. The reaction medium is anhydrous.

The reaction time is about 1 to 48 hours, a reaction time of 1 to 8 hours and particularly 2 to 6 hours being preferred.

Preferably, the compound of formula (II) and the compound of formula (III) are made to react in a 1:1 to 1:50 and particularly a 1:1 to 1:15 molar ratio. The work-up of the Grignard reaction mixture is accomplished by adding the reaction solution to an aqueous or aqueous-alcoholic solution acidified with an acid (HA), preferably to an aqueous phase saturated with NH₄Cl salt and additionally acidified with 37% HCl solution (pH 1.5, preferably 2-4). Depending on its solubility, the compound of formula (I) or (Ia) can precipitate as soon as the water has been separated, Otherwise, the aqueous phase is extracted with an organic solvent usually employed for this purpose, for example a halogenated hydrocarbon, ether, ester or supercritical CO₂ fluid, but preferably with an ether or ester and particularly with ethyl acetate. The extract thus obtained is then taken up into an aqueous-alcoholic solution acidified with an acid and allowed to agitate at a temperature of 0° to 100° C., a temperature of 200 to 50° C. being particularly preferred, The agitation is preferably allowed to last 1 to 48 hours and particularly 1 to 8 hours, This gives the compound of formula (I) or (Ia).

Suitable compounds of formulas (I) and (Ia) are in particular 7-hydroxy-4-methylene-2-phenyl-4H-1-benzopyran, 7-methoxy-4-methylene-2-(3,4,5-trimethoxyphenyl)-4H-1-benzopyran, 6,7-dihydroxy-4-methylene-2-phenyl-4H-1-benzopyran, 5,7-dimethoxy-4-methylene-2-phenyl-4H-1-benzopyran, 4-methylene-4H-1-benzopyran, 4-methylene-2-phenyl-4H-1-benzopyran, 7-amino-4-methylene-2-phenyl-4H-1-benzopyran, 7-dimethylamino-4-methylene-2-phenyl-4H-1-benzopyran and 4-methylene-7-nitro-2-phenyl-4H-1-benzopyran as well as their salts with inorganic or organic acids. Particularly preferred are 7-hydroxy-4-methyl-2-phenyl-1-benzopyrylium chloride, 7-methoxy-4-methyl-2-(3,4,5-trimethoxyphenyl)-1-benzopyrylium chloride, 5,7-dihydroxy-4-methyl-2-phenyl-1-benzopyrylium chloride, 5,7-dimethoxy-4-methyl-2-phenyl-1-benzopyrylium chloride, 4-methyl-1-benzopyrylium chloride, 4-methyl-2-phenyl-1-benzopyrylium chloride, 7-amino-4-methyl-2-phenyl-1-benzopyrylium chloride, 7-dimethylamino-4-methyl-2-phenyl-1-benzopyrylium chloride and 4-methyl-7-nitro-2-phenyl-1-benzopyrylium chloride.

The present invention concerns a method for producing cationic dyes of formulas (V) and (VI) whereby the aforedescribed 4-alkylene-2-aryl-4H-1-benzopyran derivative of formula (Ia) or a salt thereof of formula (I) is made to react with an aldehyde derivative of formula (IV), preferably 4-N,N-dimethylaminobenzaldehyde or 4-N,N-dimethylaminocinnamaldehyde, at 0 to 180° C. in a polar aprotic, apolar aprotic, polar protic or apolar protic solvent

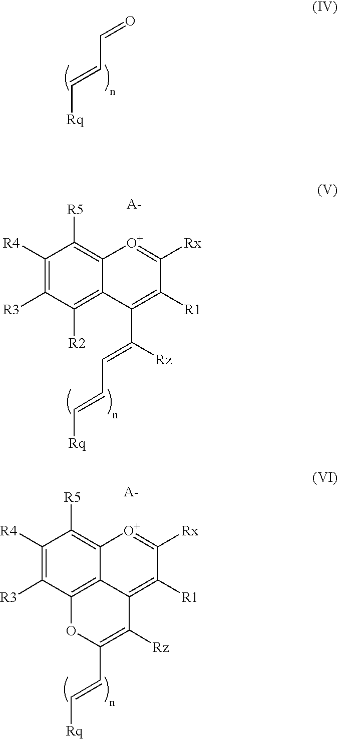

wherein

R1, R2, R3, R4 and R5 independently of each other denote a hydrogen atom, a straight-chain or branched C₁-C₄-alkyl group, a C₁-C₄-hydroxyalkyl group, a hydroxyl group, a methoxy group, a benzyl group, a halogen atom (F, Cl, Br, I), a nitro group, a nitroso group, a cyano group, a trifluoromethyl group, a —CHO group, a —COR^{b} group, a —COOH group, a —CO₂R^{b} group, an —OCOR^{b} group, an —OCH₂-aryl group, an —SO₂NH₂ group, an —SO₂CHF₂ group, an —SO₂CF₃ group, an —SO₂NH₂ group, an —SO₂NHR^{b} group, an —SO₂N(R^{b})₂ group, an —SO₂R^{b} group, an —NH₂ group, an —(NH₃)+group, an —NHR^{b} group, an —(NH₂R^{b})⁺ group, an —N(R^{b})₂ group, an —[N(R^{b})₃]⁺ group, an —NHCOR^{b} group, an —NHCOOR^{b} group, a —CH₂NH₂ group, a —CH₂NHR^{b} group, a —CH₂N(R^{b})₂ group, a —CO₂CF₃ group or a —PO(OR^{b})₂ group, wherein R^{b} stands for a hydrogen atom, an optionally substituted aromatic carbon ring or heterocyclic ring or a (C₁-C₆)-alkyl group;

R_{q} denotes a substituted pyridyl group, a substituted pyrimidyl group, a phenyl group of formula (VII) or a heterocyclic group of formula (VIII)

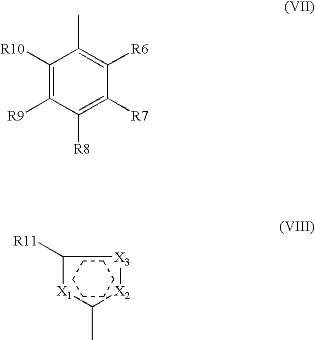

wherein

R6, R7, R8, R9 and R10 independently of each other denote a hydrogen atom, a straight-chain or branched C₁-C₄-alkyl group, a C₁-C₄-hydroxyalkyl group, a hydroxyl group, a methoxy group, a benzyl group, a halogen atom (F, Cl, Br, I), a nitro group, a nitroso group, a cyano group, a trifluoromethyl group, a —CHO group, a —COR^{c} group, a —COOH group, a —CO₂R^{c} group, an —OCOR^{c} group, an —OCH₂-aryl group, an —SO₂NH₂ group, an —NH₂ group, an —(NH₃)⁺ group, an —NHR^{c} group, an —(NH₂R^{c})⁺ group, an —N(R^{c})₂ group, an —[N(R^{c})₃]⁺ group, an —NHCOR^{c} group, an —NHCOOR^{c} group, a —CH₂NH₂ group, a —CH₂NHR^{c} group, a —CH₂N(R^{c})₂ group, a —CO₂CF₃ group, a —PO(OR^{c})₂ group, an —SO₂CHF₂ group, an —SO₂CF₃ group, an —SO₂R^{c} group or an —SR^{c} group,

wherein R^{c} denotes a hydrogen atom, an optionally substituted aromatic carbon ring or heterocyclic ring or a C₁-C₆-alkyl group, provided that two adjacent R6 to R10 groups, also together with the carbon atoms of the aromatic ring, can form a 5-membered or 6-membered alicyclic or aromatic ring which optionally can contain one or more sulfur atoms, nitrogen atoms and/or oxygen atoms; and

X1 denotes sulfur, nitrogen, oxygen, a C-R13 group or an N-R12 group;

X2 denotes sulfur, nitrogen, oxygen, a C-R14 group or an N-R12 group; and

X3 denotes sulfur, nitrogen, oxygen, a C-R15 group or an N-R12 group,

provided that at least one and at the most two of the X1 to X3 groups denote sulfur, oxygen or an N-R12 group,

R11, R13, R14 and R15 independently of each other denote hydrogen, a halogen atom (F, Cl, Br, I), a cyano group, a C₁-C₄-alkoxy group, a C₁-C₆-alkyl group, a C₁-C₄alkyl thioether group, a mercapto group, a nitro group, an amino group, a C₁-C₄-alkylamino group, a di(C₁-C₄)-alkylamino group, a di(C₁-C₄)-hydroxyalkylamino group, a C₁-C₄-hydroxyalkylamino group, a trifluoromethyl group, a —C(O)CH₃ group, a —C—(O)CF₃ group, an —Si(CH₃)₃ group, a C₁-C₄-hydroxyalkyl group or a C₃-C₄-dihydroxyalkyl group; and

R12 denotes hydrogen, a C₁-C₆-alkyl group, a C₂-C₄-hydroxyalkyl group, a phenyl group or an acetyl group;

n equals 0, 1 or 2;

Rx denotes a hydrogen atom, an optionally substituted phenyl group, an optionally substituted naphthyl group, a benzyl group, an optionally substituted aromatic carbon ring or heterocyclic ring, a C₁-C₈-polyhydroxyalkyl group, a C₁-C₈-alkoxy-(C₁-C₈)-alkyl group, a straight-chain or branched C₁-C₆-alkyl group, an —OR^{a} group or an —SR^{a} group, wherein R^{a} denotes a hydrogen atom, an optionally substituted aromatic carbon ring or heterocyclic ring or a C₁-C₆-alkyl group;

Rz denotes a hydrogen atom, an optionally substituted phenyl group, an optionally substituted naphthyl group, an optionally substituted aromatic carbon ring or heterocyclic ring, a C₁-C₈-alkyl group, a C₁-C₈-monohydroxyalkyl group, a C₁-C₈-polyhydroxyalkyl group, a C₁-C₈-alkoxy-(C₁-C₈)-alkyl group, a halogen atom, an —OCOR^{a} group, a nitro group, a cyano group, a —COR^{a} group, a —CO—OR^{a} group, a —CO—OCF₃ group, a —CONHR^{a} group, a —CO—N(R^{a})₂ group, an —SO₂—NH₂ group, an —SO₂NHR^{a} group, an —SO₂—N(R^{a})₂ group, an —SO₂OR^{a} group or an —SO₂—R^{a} group, wherein R^{a} denotes a hydrogen atom, an optionally substituted aromatic carbon ring or heterocyclic ring or a C₁-C₆-alkyl group; and

A⁻ denotes an anion of an organic or inorganic acid, preferably a chloride, bromide, iodide, hydrogen sulfate, sulfate, toluenesulfonate, benzenesulfonate, monomethyl sulfate, hexafluorophosphate, hexafluoroantimonate, tetrafluroborate, tetraphenylborate, formate, acetate or propionate, with the chloride ion, tetrafluoroborate ion, acetate ion and hydrogen sulfate ion being particularly preferred.

The condensation reaction is preferably carried out at 20 to 140 C and particularly at 50° to 100 C, the use of a polar protic or apolar protic solvent, for example, water, an alcohol such as ethanol and methanol or a carboxylic acid such as acetic acid and formic acid being preferred. Particularly preferred is the use of ethanol and/or methanol. The reaction time is preferably about 1 to 48 hours and particularly 1 to 8 hours.

The synthesis for the dyes of formula (V) and (VI) is carried out in the presence of atmospheric oxygen. The reaction is represented by the following scheme 1.

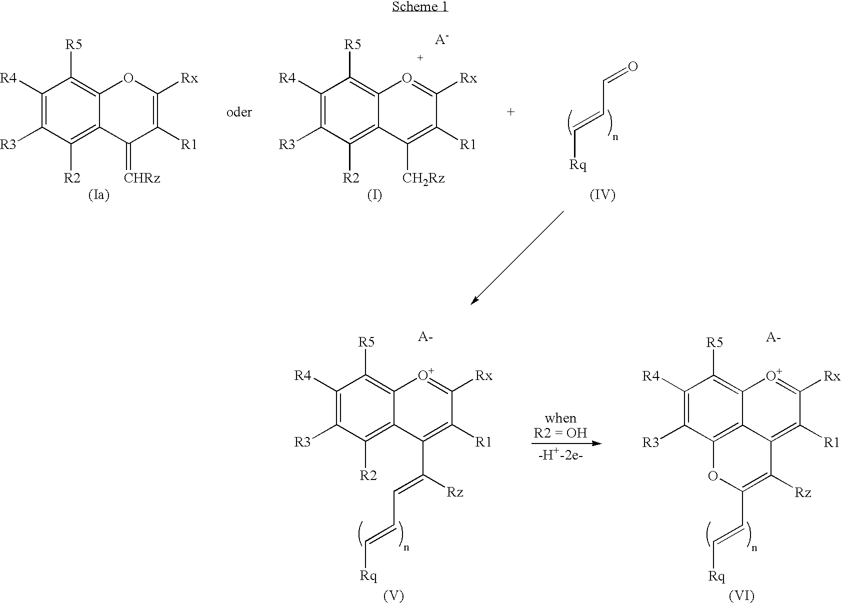

Another object of the invention are the novel cationic dyes of formula (V) and (VI).

Suitable compounds of formula (V) and (VI) are, in particular, 4-{4-[(4-N,N-dimethylamino)phenyl]ethenyl}-7-hydroxy-2-phenyl-1-benzopyrylium chloride, 4-{4-[(4-N,N-dimethylamino)phenyl]ethenyl}-7-methoxy-2-(3,4,5-trimethoxyphenyl)-1 benzopyrylium chloride, 8-hydroxy-5-phenyl-2-(4, -N,N)-dimethylamino)phenyl-1,6-dioxaphenalenium chloride, 8-hydroxy-5-phenyl-2-{2-[4-(N,N)-dimethylamino]phenyl}ethenyl-1,6-dioxaphenalenium chloride and 4-{4-[4-(N,N-dimethylamino)phenyl]-butadienyl}-7-methoxy-2-(3,4,5-trimethoxyphenyl)-1-benzopyrylium chloride.

The dyes of formula (V) and of formula (VI) are eminently suited as direct dyes for coloring keratin fibers.

Another object of the present invention therefore is an agent for dyeing keratin fibers, for example wool, cotton [sic—Translator], silk or hair, particularly human hair, characterized in that it contains at least one compound of formula (V) or (VI).

The agent of the invention for dyeing keratin fibers contains the dyes of formula (V) and (VI) in a total amount of about 0.01 to 5 wt. % and preferably 0.1 to 3 wt. %.

Moreover, the dyeing agent of the invention can, in addition, optionally contain common, physiologically harmless direct dyes from the group of acid or basic dyes, nitro dyes, azo dyes, quinone dyes and triphenylmethane dyes.

The agent of the invention for dyeing keratin fibers can be, for example, a solution, particularly an aqueous or aqueous-alcoholic solution. Other suitable formulation forms are a cream, gel, foam or emulsion. The composition of said agent consists of a mixture of compounds of formulas (V) and (VI) with additives commonly used for such formulations.

Common additives to solutions, creams, emulsions, gels or foams are, for example, solvents such as water, lower aliphatic alcohols, for example ethanol, n-propanol and isopropanol, or glycols such as glycerol and 1,2-propanediol; moreover wetting agents or emulsifiers from the classes of anionic, cationic, amphoteric or nonionic surface-active substances such as the fatty alcohol sulfates, ethoxylated fatty alcohol sulfates, alkylsulfonates, alkylbenzenesulfonates, alkyltrimethylammonium salts, alkylbetaines, ethoxylated fatty alcohols, ethoxylated nonylphenols, fatty alkanolamides, ethoxylated fatty alcohols, ethoxylated nonylphenols, fatty alkanolamides [sic—Translator], ethoxylated fatty esters, furthermore thickeners such as the higher fatty alcohols, starch or cellulose derivatives, perfumes, hair-pretreatment agents, conditioners, hair-swelling agents, preservatives, furthermore vaseline, paraffin oil and fatty acids as well as hair-care agents such as cationic resins, lanolin derivatives, cholesterol, pantothenic acid and betaine. The said constituents are used in amounts commonly employed for such purposes, for example the wetting agents and emulsifiers at a concentration of about 0.5 to 30 wt. % (based on the dye carrier composition), the thickeners in an amount of about 0.1 to 30 wt. % (based on the dye carrier composition) and the hair-care agents at a concentration of about 0.1 to 5 wt. % (based on the dye carrier composition).

It is also possible to dispense this agent from a pressurized container as an aerosol spray or aerosol foam by means of an atomizer or some other appropriate pumping device or spray system or in admixture with a common propellant liquefied under pressure.

The pH of the colorant of the invention is about 2 to 11, a pH of 2.5 to 8 being particularly preferred. The pH can be adjusted to an alkaline value with ammonia, although it is also possible to use in place of ammonia an organic amine, for example monoethanolamine or triethanolamine. To adjust the pH to an acidic value, on the other hand, an organic or inorganic acid can be used, for example hydrochloric acid, sulfuric acid, phosphoric acid, ascorbic acid, glycolic acid or lactic acid.

Of course, the aforedescribed colorant can optionally contain other additives commonly employed for keratin fibers, for example care agents, wetting agents, thickeners, softeners, preservatives and perfume oils as well as other additives indicated in the following.

The colorants of the invention can also contain wetting agents or emulsifiers from the classes of anionic, amphoteric, nonionic or zwitterionic surface-active substances such as the fatty alcohol sulfates, alkanesulfonates, alkylbenzenesulfonates, alkylbetaines, α-olefinsulfonates, ethoxylated fatty alcohols, ethoxylated nonylphenols, fatty alkanolamides, ethoxylated fatty esters, fatty alcohol polyglycol ether sulfates, alkylpolyglucosides, thickeners such as the higher fatty alcohols, starch, alginates, bentonites, cellulose derivatives, vaseline, paraffin oil and fatty acids, water-soluble polymeric thickeners such as the natural gums, guar gum, xanthan gum, carob bean meal, pectin, dextran, agar, amylose, amylopectin, dextrins, clays or fully synthetic hydrocolloids such as polyvinyl alcohol, furthermore care agents such as lanolin derivatives, cholesterol, pantothenic acid, water-soluble polymers, protein derivatives, provitamins, vitamins, plant extracts, sugar and betaine, auxiliary agents such as moisturizers, electrolytes, antioxidants, fatty amides, sequestering agents, film-forming agents and preservatives.

The aforedescribed colorant can also contain natural or synthetic polymers or modified polymers of natural origin whereby the keratin fibers are strengthened and dyed at the same time. Such colorants are generally referred to as tint strengtheners or color strengtheners. Among the synthetic polymers known in the cosmetic field to be used for this purpose are, for example, polyvinylpyrrolidone, polyvinyl acetate, polyvinyl alcohol or the polyacrylic compounds such as polyacrylic acid or polymethacrylic acid, polyacrylonitrile, polyvinyl acetate and the copolymers of such compounds, for example poly(vinylpyrrolidone-vinyl acetate). Suitable among the natural polymers or modified natural polymers are, for example, chitosan (deacetylated chitin) and chitosan derivatives.

The said constituents are used in amounts usually employed for such purposes, for example the wetting agents and emulsifiers at a concentration of about 0.5 to 30 wt. %, the thickeners in an amount from about 0.1 to 25 wt. % and the care agents in an amount from about 0.1 to 5 wt. %. The aforesaid polymers can be used in the agent of the invention in an amount that is common for such agents and particularly in an amount from about 1 to 5 wt. %.

The agent of the invention for coloring keratin fibers is particularly well suited for coloring hair. To this end, the colorant of the invention is applied to hair in the usual manner in an amount sufficient for coloring the hair, in general about 50 to 150 grams. After an exposure time sufficient for hair coloring, usually about 10 to 45 min at 20° to 50° C. and preferably 15 to 30 min at about 40° C., the hair is rinsed with water, optionally washed with a shampoo and/or an aqueous solution of a weak organic acid, for example citric acid or tartaric acid, then post-rinsed and dried.

The colorant that also provides strengthening is used in the known and conventional manner by moistening the hair with the strengthener, styling the hair and then drying.

The colorant of the invention gives colorations of outstanding brightness and color depth even without the addition of an oxidant. Although the use of the aforesaid colorant without addition of an oxidant is preferred because this method is more gentle to the fibers, it is entirely possible to use the aforesaid colorant in conjunction with an oxidant, for example when simultaneous bleaching of the fibers is desired or when common oxidation dye precursors are also to be added to the colorant.

As far as the scope of coloring is concerned, depending on the kind and composition of the dyes used, the colorants of the invention provide a wide range of different color shades ranging from natural color shades to the highly fashionable, highlighted shades. The excellent properties of the novel colorant manifest themselves especially on hair damaged by light and weather or on permanently waved hair. The colorations obtained in these cases are characterized in particular by their very good permanence and wash fastness.

The following examples are intended to illustrate the subject matter of the invention more closely without limiting its scope.

### EXAMPLES

### Examples 1.1 and 1.2

### Synthesis of 4-Alkyl-2-aryl-1-benzopyrylium salts of formula (Ia)

### Example 1.1

### 5,7-Dihydroxy-4-methyl-2-phenyl-1-benzopyrylium chloride

A solution of 1.08 g of 5,7-dihydroxyflavone in 30 mL of dry tetrahydrofuran (THF) was added slowly and dropwise to 9 mL of a 3 M methylmagnesium bromide solution in THF. The reaction solution was then heated at reflux for 2 hours after which it was added slowly to 100 mL of a saturated NH₄Cl solution, while maintaining pH 3-5 by addition of 37% HCl solution. This gave a red solid which was filtered off, washed with 150 mL of water and then dried.

Yield: 0.80 g (65% of the theoretical) of 5,7-dihydroxy-4-methyl-2-phenyl-1-benzopyrylium chloride.

¹H-NMR (CD₃OD): δ=8.40 ppm (dd, J=1.5 Hz; J=7.5 Hz, 2H); 8.16 ppm (s, 1H); 7.83-7.76 (m, 3H); 7.04 (d, J=2 Hz, 1H); 6.77 ppm (d, J=2 Hz, 1H). ESI mass spectrum: M⁺=253

UV-VIS (0.1 M HCl with 1% of MeOH): 378 nm (ε=12,800±200); 436 nm (ε=9000±200.

### Example 1.2

### 7-Methoxy-4-methyl-2-(3,4,5-trimethoxyphenyl)-1-benzopyrylium Chloride

A solution of 0.4 g of 7,3′,4′,5′-tetramethoxyflavone in 15 mL of dry THF was added slowly and dropwise to 1.6 mL of a 3 M methylmagnesium bromide solution in THF. The solution was then heated at reflux for 2 hours after which it was added to 50 mL of a saturated NH₄Cl solution. The aqueous phase was extracted with ethyl acetate. The combined organic phases were dried over Na₂SO₄, filtered and evaporated to dryness. The residue obtained in this manner was dissolved in acidified methanol (methanol with 2-3 drops of 37% HCl) and the solution was stirred 1 hour at room temperature. The solvent was completely evaporated under reduced pressure which produced a red solid.

Yield: 0.42 g (95% of the theoretical) of 7-methoxy-4-methyl-2-(3,4,5-trimethoxyphenyl)-1-benzopyrylium chloride

¹H-NMR (CD₃OD): δ=8.68 ppm (s, 1H); 8.43 ppm (d, J=9 Hz, 1H); 8.03 ppm (d, J=2 Hz, 1H); 7.84 ppm (s, 2H); 7.59 ppm (dd, J=9 Hz; J=2 Hz, 1H); 4.22 ppm (s, 3H); 4.08 (s, 6H); 4.00 ppm (s, 3H);

High-resolution mass spectrum:

M⁺ calculated for C₂₀H₂₁O₅: 341.1389; found: 341.1417

UV-VIS: (0.1 M HCl with 1% of MeOH): 444 nm (ε=28,200±500).

### Examples 2.1 to 2.5

### Synthesis of Cationic Dyes of Formulas (V) and (VI)

### Example 2.1

4-{4-[4-(N,N-dimethylamino)phenyl]ethenyl}-7-hydroxy-2-phenyl-1-benzopyrylium Chloride

0.40 g of 7-hydroxy-4-methyl-2-phenyl-1-benzopyrylium chloride and 0.24 g of 4-N,N-dimethylaminobenzaldehyde were dissolved in methanol and heated 3 hours at reflux. The reaction mixture was then evaporated to dryness. The resulting green residue was washed with diethyl ether/ethyl acetate (1:1) and recrystallized from ethanol.

Yield: 0.50 g (84% of the theoretical) of 4-{4-[4-(N,N-dimethylamino)phenyl]ethenyl}-7-hydroxy-2-phenyl-1-benzopyrylium chloride

¹H-NMR (CO₃OD): δ=8.42 ppm (d, J=15 Hz, 1H); 8.39 ppm (d, J=9 Hz, 1H); 8.25 ppm (d, J=7.5 Hz, 2H); 8.14 ppm (s, 1H); 7.87 ppm (d, J=9 Hz, 2H); 7.76 ppm (d, J=15 Hz, 1H); 7.72-7.61 ppm (m, 3H); 7.07 ppm (d, J=9 Hz, 1H); 6.97 ppm (s, 1H); 6.86 ppm (d, J=9 Hz, 2H); 3.17 ppm (s, 6H).

High-resolution mass spectrum:

M⁺ calculated for C₂₅H₂₂NO₂: 368.1650; found: 368.1654

UV-VIS: (MeOH with 5% of 0.1 M HCl): 640 nm (ε=80,000±1000);

### Example 2.2

### 4-{4-[4-(N,N-Dimethylamino)phenyl]ethenyl}-7-methoxy-2-(3,4,5-trimethoxyphenyl)-1-benzopyrylium Chloride

0.18 g of 7-methoxy-4-methyl-2-(3,4,5-trimethoxyphenyl)-1-benzopyrylium chloride and 0.078 g of 4-N,N-dimethylaminobenzaldehyde were dissolved in methanol and heated at reflux for three hours. The reaction mixture was then evaporated to dryness. The green residue thus obtained was washed with diethyl ether/ethyl acetate (1/1) and recrystallized from ethanol.

Yield: 0.20 g (80% of the theoretical) of 4-{4-[4-(N,N-dimethylamino)phenyl]ethenyl}7-methoxy-2-(3,4,5-trimethoxyphenyl)-1-benzopyrylium chloride.

¹H-NMR (CD₃OD): δ=8.69 ppm (d, J=15 Hz, 1H); 8.47 ppm (d, J=9 Hz, 1H); 8.25 ppm (s, 1H); 7.95 ppm (d, J=9 Hz, 2H); 7.72 ppm (d, J=15 Hz, 1H); 7.58 ppm (s, 2H); 7.51 ppm (d, J=2 Hz, 1H); 7.28 ppm (dd, J=9 Hz; J=2 Hz, 1H); 6.92 ppm (d, J=9 Hz, 2H); 4.08 ppm (s, 3H); 4.06 ppm (s, 6H); 3.95 ppm (s, 3H); 3.29 ppm (s, 6H);

High-resolution mass spectrum:

M⁺ calculated for C₂₉H₃₀NO₅: 472.2124; found 472.2125

UV-VIS: (MeOH with 5% of 0.1 M HCl): 652 (ε=78,600±800).

### Example 2.3

### 8-Hydroxy-5-phenyl-2-[4-(N,N-dimethylamino)phenyl]-1,6-dioxaphenalenium Chloride

0.58 g of 5,7-dihydroxy-4-methyl-2-phenyl-1-benzopyrylium chloride and 0.33 g of 4-N,N-dimethylaminobenzaldehyde in 30 mL of methanol were heated at reflux for three hours. The reaction mixture was then evaporated to dryness. The green residue thus obtained was washed once with diethyl ether/ethyl acetate (1/1) and recrystallized from ethanol.

Yield: 0.47 g (56% of the theoretical) of 8-hydroxy-5-phenyl-2-[4-(N,N-dimethylamino)phenyl]-1,6-dioxaphenalenium chloride

¹H-NMR (CD₃OD): δ=7.97 ppm (d, J=7.5 Hz, 2H); 7.78 ppm (d, J=9 Hz, 2H); 7.61 ppm (m, 1H); 7.53 ppm (m, 2H); 7.25 ppm (s, 1H); 7.18 ppm (s, 1H); 6.89 ppm (d, J=2 Hz, 1H); 6.83 ppm (d, J=2 Hz, 1H); 6.62 ppm (d, J=9 Hz, 2H); 3.00 ppm (s, 6H).

High-resolution mass spectrum:

M⁺ calculated for C₂₅H₂₀NO₃: 382.1443; found 382.1443.

UV-VIS: (MeOH with 5% of 0.1 M HCl): 548 nm (ε=46,500±500).

### Example 2.4

### 8-Hydroxy-5-phenyl-2-{2-[4-(N,N-dimethylamino)phenyl]}ethenyl-1,6-dioxaphenalenium Chloride

0.55 g of 5,7-dihydroxy-4-methyl-2-phenyl-1-benzopyrylium chloride and 0.37 g of 4-N,N-dimethylaminocinnamaldehyde in 30 mL of methanol were heated at reflux for 7 hours. The methanol was then completely evaporated. The green residue thus obtained was washed once with diethyl ether/ethyl acetate (1/1) and once with CHCl₃/ethyl acetate (1/1) affording a dark-red solid.

Yield: 0.75 g (89% of the theoretical) of 8-hydroxy-5-phenyl-2-{2-[4-(N,N-dimethylamino)phenyl]-}ethenyl-1,6-dioxaphenalenium chloride.

¹H-NMR (CD₃OD): δ=7.99 ppm (d, J=7.5 Hz, 2H); 7.67-7.54 ppm (m, 4H); 7.36 ppm (d, J=9 Hz, 2H); 7.26 ppm (s, 1H); 6.96 ppm (d, J=2 Hz, 1H); 6.93 ppm (d, J=2 Hz, 2H); 6.73 ppm (s, 1H); 6.69 ppm (d, J=15 hz, 1H); 6.54 ppm (d, J=9 Hz, 2H); 2.94 ppm (s, 6H).

High-resolution mass spectrum:

M⁺ calculated for C₂₇H₂₂NO₃: 408.1600; found 408.1600

UV-VIS: (MeOH with 5% of 0.1 M HCl): 628 nm (ε=19,100±300).

### Example 2.5

### 4-{4-[4-(N,N-Dimethylamino)phenyl]butadienyl}-7-methoxy-2-(3,4,5-trimethoxyphenyl)-1-benzopyrylium Chloride

0.15 g of 7-methoxy-4-methyl-2-(3,4,5-trimethoxyphenyl)-1-benzopyrylium chloride and 0.077 g of 4-N,N-dimethylaminocinnamaldehyde were heated in methanol at reflux for 3 hours. The reaction mixture was then evaporated to dryness. The green residue thus obtained was purified by chromatography on silica gel with MeOH/CH₂Cl₂ (5/95) as eluent.

Yield: 0.16 g (75% of the theoretical) of 4-{4-[4-(N,N-dimethylamino)phenyl]butadienyl}-7-methoxy-2-(3,4,5-trimethoxyphenyl)-1-benzopyrylium chloride.

¹H-NMR (CD₃OD with CF₃COOD): δ=8.62 ppm (dd, J=15 Hz, J=12 Hz, 1H); 8.39 ppm (d, J=9 Hz, 1H); 8.38 ppm (s, 1H); 7.72 ppm (d, J=9 Hz, 2H); 7.65 ppm (s, 2H); 7.61 ppm (d, J=15 Hz, 1H); 7.59 ppm (d, J=2 Hz, 1H); 7.53 ppm (d, J=15 Hz, 1H); 7.45 ppm (dd, J=15 Hz, J=12 Hz, 1H); 7.34 ppm (dd, (J=9 Hz, J=2 Hz, 1H); 7.10 ppm (d, J=9 Hz, 2H); 4.07 ppm (s, 3H); 4.03 ppm (s, 6H); 3.92 ppm (s, 3H); 3.21 ppm (s, 6H).

High-resolution mass spectrum:

M⁺ calculated for C₃₁H₃₂NO₅: 498.2280; found: 498.2291.

UV-VIS (MeOH with 5% of 0.1 M HCl): 730 nm (ε=66,600±900)

### Examples 3.1 to 3.10

### Colorants for Keratin Fibers

| Example 3.1 | Hair Colorant |
|---|---|
| 0.05 g | of 4-{4-[4-(N,N-dimethylamino)phenyl]ethenyl}-7- |
| | hydroxy-2-phenyl-1-benzopyrylium chloride |
| 50.00 g | of ethanol |
| to 100.00 g | water, demineralized |

By addition of 10% citric acid solution or 0.1 M NaOH solution, the above colorant solution was adjusted to the pHₘ indicated for Examples 3.1. a-d and then applied to bleached hair. After an exposure time of 30 min at 40° C., the hair was washed with water and dried.

| | | Shade | | | | |
|---|---|---|---|---|---|---|
| Example | | After | | Color Values | | |
| No. | | Dyeing | | L | a | b |
| | | | untreated | 63.30 | −0.48 | +10.40 |
| | | | hair: | | | |
| 3.1.a | pHₘ 4.9 | violet | after dyeing: | 31.10 | +7.61 | −8.41 |
| 3.1.b | pHₘ 3.2 | violet | after dyeing: | 34.89 | +7.08 | −10.89 |
| 3.1.c | pHₘ 6.4 | violet | after dyeing: | 32.51 | +9.00 | −11.06 |
| 3.1.d | pHₘ 9.1 | violet | after dyeing: | 33.61 | +8.60 | −10.75 |

| Example 3.2 | Hair Colorant |
|---|---|
| 0.20 g | of 4-{4-[4-(N,N-dimethylamino)phenyl]ethenyl}- |
| | 7-hydroxy-2-phenyl-1-benzopyrylium chloride |
| 1.60 g | of decylglucoside (Plantacare 2000 UP, by Fluka) |
| 0.08 g | of disodium ethylenediaminetetraacetate |
| 62.00 g | of ethanol |
| to 100.00 g | water, demineralized |

The above coloring solution was applied to bleached hair (L=63.3; a=−0.48; b=10.4). After an exposure time of 30 min at 40° C., the hair was washed with water and dried. A violet coloration was obtained (L=33.97; a=7.35; b=−10.88).

| Example 3.3 | Hair Colorant |
|---|---|
| 0.05 g | of 4-{4-[4-(N,N-dimethylamino)phenyl]ethenyl}- |
| | 7-methoxy-2-(3,4,5-trimethoxyphenyl)-1-benzopyrylium |
| | chloride |
| 50.00 g | of ethanol |
| to 100.00 g | water, demineralized |

The above coloring solution was applied to bleached hair (L=63.3; a=−0.48; b=10.4). After an exposure time of 30 min at 40° C., the hair was washed with water and dried. A blue-green coloration was obtained (L=39.22; a=−10.15; b=−8.19).

| Example 3.4 | Hair Colorant |
|---|---|
| 0.320 g | of 4-{4-[4-(N,N-dimethylamino)phenyl]ethenyl}- |
| | 7-methoxy-2-(3,4,5-trimethoxyphenyl)-1-benzopyrylium |
| | chloride |
| 1.410 g | of cocoamidopropylbetaine |
| 0.014 g | of formic acid |
| 40.600 g | of ethanol |
| to 100.000 g | water, demineralized |

The above coloring solution was applied to bleached hair (L=63.3; a=−0.48; b=10.4). After an exposure time of 30 min at 40° C., the hair was washed with water and dried. A violet coloration was obtained (L=39.50; a=2.19; b=−8.31).

| Example 3.5 | Hair Colorant |
|---|---|
| 0.05 g | of 4-{4-[4-(N,N-dimethylamino)phenyl]butadienyl}- |
| | 7-methoxy-2-(3,4,5-trimethoxyphenyl)-1-benzopyrylium |
| | chloride |
| 50.00 g | of ethanol |
| to 100.00 g | water, demineralized |

The above coloring solution was applied to bleached hair (L=63.3; a=−0.48; b=10.4). After an exposure time of 30 min at 40° C., the hair was washed with water and dried. A green coloration was obtained (L=42.26; a=−20.94; b=0.59).

| Example 3.6 | Hair Colorant |
|---|---|
| 0.27 g | of 4-{4-[4-(N,N-dimethylamino)phenyl]butadienyl}- |
| | 7-methoxy-2-(3,4,5-trimethoxyphenyl)-1-benzopyrylium |
| | chloride |
| 2.00 g | of decylglucoside (Plantacare 200 UP, by Fluka) |
| 0.10 g | of disodium ethylenediaminetetraacetate |
| 52.50 g | of ethanol |
| to 100.00 g | water, demineralized |

The above coloring solution was applied to bleached hair (L=63.3; a=−0.48; b=10.4). After an exposure time of 30 min at 40° C., the hair was washed with water and dried. A blue-green coloration was obtained (L=35.40; a=−18.42; b=2.12).

| | |
|---|---|
| Example 3.7 | Hair Colorant |

| | |
|---|---|
| 0.01 g | of 7-hydroxy-2-phenyl-4-[(E)-2-(2-thienyl)ethenyl]-1- |
| | benzopyrylium chloride |
| 50.00 g | of ethanol |
| to 100.00 g | water, demineralized |

By addition of 10% citric acid solution or 0.1 M NaOH solution, the above colorant solution was adjusted to the pHₘ indicated for Examples 3.7. a-d and then applied to bleached hair. After an exposure time of 30 min at 40° C., the hair was washed with water and dried.

| | | Shad | | | | |
|---|---|---|---|---|---|---|
| Example | | After | | Color Valu s | | |
| No. | | Dyeing | | L | a | b |
| | | | untreated | 63.30 | −0.48 | +10.40 |
| | | | hair | | | |
| 3.7.a | pHₘ 3.8 | orange | after | 66.64 | +21.24 | +19.15 |
| | | | dyeing | | | |
| 3.7.b | pHₘ 2.9 | orange | after | 63.55 | +25.43 | +23.55 |
| | | | dyeing | | | |
| 3.7.c | pHₘ 6.0 | orange | after | 68.75 | +19.44 | +18.93 |
| | | | dyeing | | | |
| 3.7.d | pHₘ 9.6 | orange | after | 67.97 | +21.08 | +19.15 |
| | | | dyeing | | | |

| Example 3.8 | Hair Colorant |
|---|---|
| 0.01 g | of 4-[(E)-2-(2-furyl)ethenyl]-7-hydroxy-2-phenyl-1- |
| | benzopyrylium chloride |
| 50.00 g | of ethanol |
| to 100.00 g | water, demineralized |

By addition of 10% citric acid solution or 0.1 M NaOH solution, the above colorant solution was adjusted to the pHₘ indicated for Examples 3.8. a-d and then applied to bleached hair. After an exposure time of 30 min at 40° C., the hair was washed with water and dried.

| | | Shade | | | | |
|---|---|---|---|---|---|---|
| Example | | After | | Color Values | | |
| No. | | Dyeing | | L | a | b |
| | | | untreated | 63.30 | −0.48 | +10.40 |
| | | | hair | | | |
| 3.8.a | pHₘ 3.6 | orange | after | 60.08 | +19.60 | +19.55 |
| | | | dyeing | | | |
| 3.8.b | pHₘ 2.8 | orange | after | 51.41 | +24.76 | +24.30 |
| | | | dyeing | | | |
| 3.8.c | pHₘ 6.0 | orange | after | 66.81 | +19.33 | +19.52 |
| | | | dyeing | | | |
| 3.8.d | pHₘ 9.5 | orange | after | 64.94 | +19.40 | +18.62 |
| | | | dyeing | | | |

| Example 3.9 | Hair Colorant |
|---|---|
| 0.01 g | of 4-[(E)-2-(2,4-dihydroxyphenyl)ethenyl]-7-hydroxy- |
| | 2-phenyl-1-benzopyrylium chloride |
| 50.00 g | of ethanol |
| to 100.00 g | water, demineralized |

By addition of 10% citric acid solution or 0.1 M NaOH solution, the above colorant solution was adjusted to the pHₘ indicated for Examples 3.9. a-d and then applied to bleached hair. After an exposure time of 30 min at 40° C., the hair was washed with water and dried.

| | | Shade | | | | |
|---|---|---|---|---|---|---|
| Example | | After | | Color Values | | |
| No. | | Dyeing | | L | a | b |
| | | | untreated | 63.30 | −0.48 | +10.40 |
| | | | hair | | | |
| 3.9.a | pHₘ 4.0 | red | after | 58.34 | +14.71 | +6.44 |
| | | | dyeing | | | |
| 3.9.b | pHₘ 3.0 | red | after | 36.63 | +15.13 | +2.70 |
| | | | dyeing | | | |
| 3.9.c | pHₘ 6.1 | red | after | 65.38 | +12.78 | +6.52 |
| | | | dyeing | | | |
| 3.9.d | pHₘ 9.1 | red | after | 54.45 | +8.24 | +2.77 |
| | | | dyeing | | | |

| Example 3.10 | Hair Colorant |
|---|---|
| 0.01 g | of 8-hydroxy-5-phenyl-2-[4-(N,N-dimethylamino)- |
| | phenyl]-1,6-dioxaphenalenium chloride |
| 50.00 g | of ethanol |
| to 100.00 g | water, demineralized |

By addition of 10% citric acid solution or 0.1 M NaOH solution, the above colorant solution was adjusted to the pHₘ indicated for Examples 3.10. a-d and then applied to bleached hair. After an exposure time of 30 min at 40° C., the hair was washed with water and dried.

| | | Shade | | | | |
|---|---|---|---|---|---|---|
| Example | | After | | Color Values | | |
| No. | | Dyeing | | L | a | b |
| | | | untreated | 63.30 | −0.48 | +10.40 |
| | | | hair | | | |
| 3.10.a | pHₘ 4.5 | wine- | after | 34.92 | +15.81 | +1.44 |
| | | red | dyeing | | | |
| 3.10.b | pHₘ 3.2 | wine- | after | 33.81 | +16.82 | −1.30 |
| | | red | dyeing | | | |
| 3.10.c | pHₘ 5.6 | wine- | after | 35.08 | +16.48 | +0.45 |
| | | red | dyeing | | | |
| 3.10.d | pHₘ 9.9 | wine- | after | 51.81 | +16.30 | +0.48 |
| | | red | dyeing | | | |

Unless otherwise indicated, all percentages are by weight.

1. Cationic dye of formula (V) or (VI) 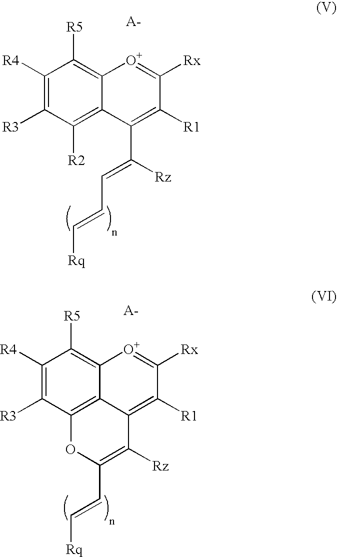
wherein
R1, R2, R3, R4 and R5 independently of each other denote a hydrogen atom, a straight-chain or branched C₁-C₄-alkyl group, a C₁-C₄-hydroxyalkyl group, a hydroxyl group, a methoxy group, a benzyl group, a halogen atom, a nitro group, a nitroso group, a cyano group, a trifluoromethyl group, a —CHO group, a —COR^{b} group, a —COOH group, a —CO₂R^{b} group, an —OCOR^{b} group, an —OCH₂-aryl group, an —SO₂NH₂ group, an —SO₂CHF₂ group, an —SO₂CF₃ group, an-SO₂NH₂ group, an —SO₂NHR^{b} group, an —SO₂N(R^{b})₂ group, an-SO₂R^{b} group, an —NH₂ group, an —(NH₃)⁺ group, an —NHR^{b} group, an —(NH₂R^{b})⁺ group, an —N(R^{b})₂ group, an —[N(R^{b})₃]⁺ group, an —NHCOR^{b} group, an —NHCOOR^{b} group, a —CH₂NH₂ group, a —CH₂NHR^{b} group, a —CH₂N(R^{b})₂ group, a —CO₂CF₃ group or a —PO(OR^{b})₂ group, wherein R^{b} stands for a hydrogen atom, an optionally substituted aromatic carbon ring or heterocyclic ring or a (C₁-C₆)-alkyl group;
R^{q} denotes a substituted pyridyl group, a substituted pyrimidyl group, a phenyl group of formula (VII) or a heterocyclic group of formula (VIII) 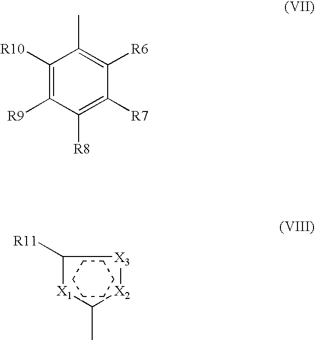
wherein
R6, R7, R8, R9 and R10 independently of each other denote a hydrogen atom, a straight-chain or branched C₁-C₄-alkyl group, a C₁-C₄-hydroxyalkyl group, a hydroxyl group, a methoxy group, a benzyl group, a halogen atom, a nitro group, a nitroso group, a cyano group, a trifluoromethyl group, a —CHO group, a —COR^{c} group, a —COOH group, a —CO₂R^{c} group, an OCOR^{c} group, an —OCH₂-aryl group, an —SO₂NH₂ group, an —NH₂ group, an —(NH₃)⁺ group, an —NHR^{c} group, an —(NH₂R^{c})⁺ group, an —(N(R^{c})₂ group, an —[N(R^{c})₃]⁺ group, an —NHCOR^{c} group, an NHCOOR^{c} group, a —CH₂NH₂ group, a —CH₂NHR^{c} group, a CH₂N(R^{c})₂ group, a —CO₂CF₃ group, a PO(OR^{c})₂ group, an —SO₂CHF₂ group, an —SO₂CF₃ group, an —SO₂R^{c} group or an —SR^{c} group,
wherein R^{c} denotes a hydrogen atom, an optionally substituted aromatic carbon ring or heterocyclic ring or a C₁-C₆-alkyl group, provided that two adjacent R6 to R10 groups, also together with the carbon atoms of the aromatic ring, can form a 5-membered or 6-membered alicyclic or aromatic ring which optionally can contain one or more sulfur atoms, nitrogen atoms and/or oxygen atoms; and
X1 denotes sulfur, nitrogen, oxygen, a C-R13 group or an N-R12 group;
X2 denotes sulfur, nitrogen, oxygen, a C-R14 group or an N-R12 group; and
X3 denotes sulfur, nitrogen, oxygen or a C-R15 group or an N-R12 group,
provided that at least one or at the most two of the X1 to X3 groups denote sulfur, oxygen or an N-R12 group,
R11, R13, R14 and R15 independently of each other denote hydrogen, a halogen atom (F, Cl, Br, I), a cyano group, a C₁-C₄-alkoxy group, a C₁-C₆-alkyl group, a C₁-C₄alkyl thioether group, a mercapto group, a nitro group, an amino group, a C₁-C₄alkylamino group, a di(C₁-C₄)-alkylamino group, a di(C₁-C₄-hydroxyalkyl)amino group, a C₁-C₄-hydroxyalkylamino group, a trifluoromethyl group, a —C(O)CH₃ group, a —C—(O)CF₃ group, an —Si(CH₃)₃ group, a C₁-C₄-hydroxyalkyl group or a C₃-C₄-dihydroxyalkyl group; and
R12 denotes hydrogen, a C₁-C₆-alkyl group, a C₂-C₄-hydroxyalkyl group, a phenyl group or an acetyl group;
n equals 0, 1 or 2;
Rx denotes a hydrogen atom, an optionally substituted phenyl group, an optionally substituted naphthyl group, a benzyl group, an optionally substituted aromatic carbon ring or heterocyclic ring, a C₁-C₈-polyhydroxyalkyl group, a C₁-C₈-alkoxy-(C₁-C₈)-alkyl group, a straight-chain or branched C₁-C₈-alkyl group, an —OR^{a} group or an —SR^{a} group, wherein R^{a} denotes a hydrogen atom, an optionally substituted aromatic carbon ring or heterocyclic ring or a C₁-C₆-alkyl group;
Rz denotes a hydrogen atom, an optionally substituted phenyl group, an optionally substituted naphthyl group, an optionally substituted aromatic carbon ring or heterocyclic ring, a C₁-C₈-alkyl group, a C₁-C₈-monohydroxyalkyl group, a C₁-C₈-polyhydroxyalkyl group, a C₁-C₈-alkoxy-(C₁-C₈)-alkyl group, a halogen atom, an —OCOR^{a} group, a nitro group, a cyano group, a —CO—R^{a} group, a —CO—OR^{a} group, a —COOCF₃ group, a —CONHR^{a} group, a —CO—N(R^{a})₂ group, an —SO₂—NH₂ group, an —SO₂NHR^{a} group, an —SO₂—N(R^{a})₂ group, an —SO₂—OR^{a} group or an —SO₂—R^{a} group, wherein R^{a} denotes a hydrogen atom, an optionally substituted aromatic carbon ring or heterocyclic ring or a C₁-C₆-alkyl group; and
A⁻ denotes an anion of an organic or inorganic acid.

2. Cationic dye according to claim 1, characterized in that it is selected from among 4-{[4-(4-N,N-dimethylamino)phenyl]ethenyl}-7-hydroxy-2-phenyl-1-benzopyrylium chloride, 4-{[4-(4-N,N-dimethylamino)phenyl]ethenyl}-7-methoxy-2-(3,4,5-trimethoxyphenyl)-1-benzopyrylium chloride, 8-hydroxy-5-phenyl-2-(4-N,N-dimethylamino)phenyl-1,6-dioxaphenalenium chloride, 8-hydroxy-5-phenyl-2-{2-[4-(N,N-dimethylamino)phenyl]ethenyl}-1,6-dioxaphenalenium chloride and 4-{4-[4-(N,N-dimethylamino)phenyl]butadienyl}-7-methoxy-2-(3,4,5-trimethoxyphenyl)-1-benzopyrylium chloride.

3. Method for producing cationic dyes of formula (V) or (VI) according to claims 1 or 2 whereby a 4-alkylene-2-aryl-4H-1-benzopyran derivative of formula (Ia) or a salt thereof of formula (I) 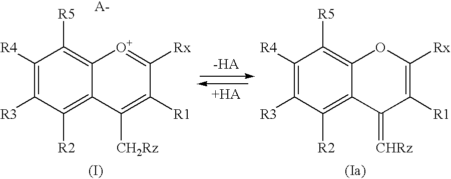
is made to react with an aldehyde derivative of formula (IV) at 0° to 180° C. in a polar aprotic, apolar aprotic, polar protic or apolar protic solvent 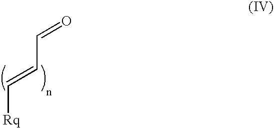
wherein
R1, R2, R3, R4 and R5 independently of each other denote a hydrogen atom, a straight-chain or branched C₁-C₄-alkyl group, a C₁-C₄-hydroxyalkyl group, a hydroxyl group, a methoxy group, a benzyl group, a halogen atom (F, Cl, Br, I), a nitro group, a nitroso group, a cyano group, a trifluoromethyl group, a —CHO group, a —COR^{b} group, a —COOH group, a —CO₂R^{b} group, an —OCOR^{b} group, an —OCH₂-aryl group, an —SO₂NH₂ group, an —SO₂CHF₂ group, an —SO₂CF₃ group, an —SO₂NH₂ group, an —SO₂NHR^{b} group, an —SO₂N(R^{b})₂ group, an —SO₂R^{b} group, an —NH₂ group, an —(NH₃)⁺ group, an —NHR^{b} group, an —(NH₂R^{b})⁺ group, an —N(R^{b})₂ group, an —[N(R^{b})₃]⁺ group, an —NHCOR^{b} group, an —NHCOOR^{b} group, a —CH₂NH₂ group, a —CH₂NHR^{b} group, a —CH₂N(R^{b})₂ group, a —CO₂CF₃ group or a —PO(OR^{b})₂ group, wherein R^{b} stands for a hydrogen atom, an optionally substituted aromatic carbon ring or heterocyclic ring or a (C₁-C₆)-alkyl group;
R^{q} denotes a substituted pyridyl group, a substituted pyrimidyl group, a phenyl group of formula (VII) or a heterocyclic group of formula (VIII) 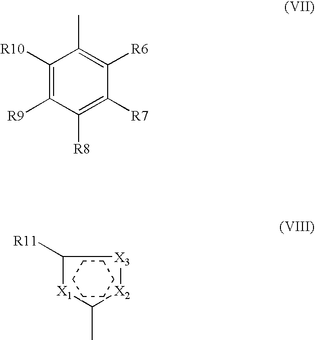
wherein
R6, R7, R8, R9 and R10 independently of each other denote a hydrogen atom, a straight-chain or branched C₁-C₄-alkyl group, a C₄-C₄-hydroxyalkyl group, a hydroxyl group, a methoxy group, a benzyl group, a halogen atom (F, Cl, Br, I), a nitro group, a nitroso group, a cyano group, a trifluoromethyl group, a —CHO group, a —COR^{c} group, a —COOH group, a —CO₂R^{c} group, an —OCOR^{c} group, an —OCH₂-aryl group, an —SO₂NH₂ group, an —NH₂ group, an —(NH₃)⁺ group, an —NHR^{c} group, an —(NH₂R^{c})⁺ group, an —N(R^{c})₂ group, an —[N(R^{c})₃]⁺ group, an —NHCOR^{c} group, an —NHCOOR^{c} group, a —CH₂NH₂ group, a —CH₂NHR^{c} group, a —CH₂N(R^{c})₂ group, a —CO₂CF₃ group, a —PO(OR^{c})₂ group, an —SO₂CHF₂ group, an —SO₂CF₃ group, an —SO₂R^{c} group or an —SR^{c} group, wherein R^{c} denotes a hydrogen atom, an optionally substituted aromatic carbon ring or heterocyclic ring or a C₁-C₆-alkyl group, provided that two adjacent R6 to R10 groups, also together with the carbon atoms or the aromatic ring, can form a 5-membered or 6-membered alicyclic or aromatic ring which optionally can contain one or more sulfur atoms, nitrogen atoms and/or oxygen atoms; and
X1 denotes sulfur, nitrogen, oxygen, a C-R13 group or an N-R12 group;
X2 denotes sulfur, nitrogen, oxygen, a C-R14 group or an N-R12 group; and
X3 denotes sulfur, nitrogen, oxygen or a C-R15 group or an N-R12 group, provided that at least one or at the most two of the X1 to X3 groups denote sulfur, oxygen or an N-R12 group,
R11, R13, R14 and R15 independently of each other denote hydrogen, a halogen atom (F, Cl, Br, I), a cyano group, a C₁-C₄-alkoxy group, a C₁-C₆-alkyl group, a C₁-C₄-alkyl thioether group, a mercapto group, a nitro group, an amino group, a C₁-C₄-alkylamino group, a di(C₁-C₄)-alkylamino group, a di(C₁-C₄-hydroxyalkyl)amino group, a C₁-C₄-hydroxyalkylamino group, a trifluoromethyl group, a —C(O)CH₃ group, a —C—(O)CF₃ group, an —Si(CH₃)₃ group, a C₁-C₄-hydroxyalkyl group or a C₃-C₄-dihydroxyalkyl group; and
R12 denotes hydrogen, a C₁-C₆-alkyl group, a C₂-C₄-hydroxyalkyl group, a phenyl group or an acetyl group;
n equals 0, 1 or 2;
Rx denotes a hydrogen atom, an optionally substituted phenyl group, an optionally substituted naphthyl group, a benzyl group, an optionally substituted aromatic carbon ring or heterocyclic ring, a C₁-C₈-polyhydroxyalkyl group, a C₁-C₈-alkoxy-(C₁-C₈)-alkyl group, a straight-chain or branched C₁-C₆-alkyl group, an —OR^{a} group or an —SR^{a} group, wherein R^{a} denotes a hydrogen atom, an optionally substituted aromatic carbon ring or heterocyclic ring or a C₁-C₆-alkyl group;
Rz denotes a hydrogen atom, an optionally substituted phenyl group, an optionally substituted naphthyl group, an optionally substituted aromatic carbon ring or heterocyclic ring, a C₁-C₈-alkyl group, a C₁-C₈-monohydroxyalkyl group, a C₁-C₈-polyhydroxyalkyl group, a C₁-C₈-alkoxy-(C₁-C₈)-alkyl group, a halogen atom, an —OCOR^{a} group, a nitro group, a cyano group, a —CO—R^{a} group, a —CO—OR^{a} group, a —COOCF₃ group, a —CONHR^{a} group, a —CO—N(R^{a})₂ group, an —SO₂—NH₂ group, an —SO₂—NHR^{a} group, an —SO₂—N(R^{a})₂ group, an —SO₂—OR^{a} group or an —SO₂—R^{a} group, wherein R^{a} denotes a hydrogen atom, an optionally substituted aromatic carbon ring or heterocyclic ring or a C₁-C₆-alkyl group; and
A⁻ denotes an anion of an organic or inorganic acid.

4. Method according to claim 3, characterized in that the reaction time is from 1 to 48 hours.

5. Method according to claim 3 or 4, characterized in that the 4-alkylene-2-aryl-4H-1-benzopyran derivative of formula (Ia) or a salt thereof of formula (I) is selected from among 7-hydroxy-4-methylene-2-phenyl-4H-1-benzopyran, 7-methoxy-4-methylene-2-(3,4,5-trimethoxyphenyl)-4H-1-benzopyran, 5,7-dihydroxy-4-methylene-2-phenyl-4H-1-benzopyran, 5,7-dimethoxy-4-methylene-2-phenyl-4H-1-benzopyran, 4-methylene-4H-1-benzopyran, 4-methylene-2-phenyl-4H-1-benzopyran, 7-amino-4-methylene-2-phenyl-4H-1-benzopyran, 7-dimethylamino-4-methylene-2-phenyl-4H-1-benzopyran and 4-methylene-7-nitro-2-phenyl-4H-1-benzopyran as well as the salts thereof with inorganic or organic acids.

6. Method according to one of claims 3 to 5, characterized in that the 4-alkylene-2-aryl-4H-1-benzopyran derivative of formula (Ia) or the salt thereof of formula (I) is selected from among 7-hydroxy-4-methyl-2-phenyl-1-benzopyrylium chloride, 7-methoxy-4-methyl-2-(3,4,5-trimethoxyphenyl)-1-benzopyrylium chloride, 5,7-dihydroxy-4-methyl-2-phenyl-1-benzopyrylium chloride, 5,7-dimethoxy-4-methyl-2-phenyl-1-benzopyrylium chloride, 4-methyl-1-benzopyrylium chloride, 4-methyl-2-phenyl-1-benzopyrylium chloride, 7-amino-4-methyl-2-phenyl-1-benzopyrylium chloride, 7-dimethylamino-4-methyl-2-phenyl-1-benzopyrylium chloride and 4-methyl-7-nitro-2-phenyl-1-benzopyrylium chloride.

7. Method according to one of claims 3 to 6 characterized in that the aldehyde derivative of formula (IV) is selected from among 4-N,N-dimethylaminobenzaldehyde and 4-N,N-dimethylaminocinnamaldehyde.

8. Agent for dyeing keratin fibers, characterized in that it contains in a suitable cosmetic base at least one cationic dye of formula (V) or (VI) according to claim 1 or claim 2.

9. Agent according to claim 8, characterized in that it contains the cationic dye of formula (V) or (VI) in an amount from 0.01 to 5 wt. %.

10. Agent according to claim 8 or 9, characterized in that it is a hair colorant.
